(19) United States
(12) Patent Application Publication
(10) Pub. No.: US 2016/0374898 A1
(43) Pub. Date: Dec. 29, 2016

(54) Wearable Self-Adhering Pill Container

(71) Applicant: Allyson Yates Hogan, Park City, UT (US)

(72) Inventor: Allyson Yates Hogan, Park City, UT (US)

(21) Appl. No.: 15/191,318

(22) Filed: Jun. 23, 2016

Related U.S. Application Data
(60) Provisional application No. 62/231,044, filed on Jun. 24, 2015.

(51) Int. Cl. A61J 1/03; B65D 75/58; B65D 75/30; A61M 5/00; B65D 65/14

(52) U.S. Cl. CPC A61J 1/035; A61M 5/002; B65D 65/14; B65D 75/30; B65D 75/5855

(57) ABSTRACT

A wearable self-adhering drug dosage unit container includes: a substrate member; a container member attached to a top side of the substrate member so as to be hermetically sealed and form a container having a fluid tight internal chamber, the internal chamber housing at least one drug dosage unit; a medically acceptable adhesive layer on a bottom side of the substrate member, the medical adhesive layer having a medically acceptable adhesive capable of adhering the container to skin; and a releasable liner on the adhesive layer. A method of administering a drug to a subject includes: providing a wearable self-adhering drug dosage unit container; adhering the container to skin by contacting the adhesive layer to the skin; wearing the container on the skin; removing the container from the skin upon initiation of an acute medical event; removing the drug dosage unit from the container; and administering the drug.

### CROSS-REFERENCE

This patent application claims priority to U.S. Provisional Application 62/231,044 filed Jun. 24, 2015, which provisional application is incorporated herein by specific reference in its entirety.

### BACKGROUND

Persons with certain medical issues need to have their medications, such as pills, tablets, and capsules, readily accessible at all times. For example, someone who has suffered a heart attack may have an immediate need for an aspirin and/or nitroglycerin tablet when heart pains occur. In this respect, in general, it would be desirable for a person to have a container with their medications readily accessible regardless of where they are or what possessions they have with them.

If a person's medications are contained in a conventional bottle, carrying the bottle at all times may be very inconvenient. One aspect of the inconvenience of a bottle is that a person may not have adequate pockets or room in a purse to carry it. Another aspect of the inconvenience of a bottle is that it can be easily misplaced. In view of the above, it would be desirable if a container for a person's medications were provided which is both readily accessible to the person and eliminates the inconvenience of having to carry a traditional pill container.

### SUMMARY

In one embodiment, a wearable self-adhering drug dosage unit container can include: a substrate member; a container member attached to a top side of the substrate member so as to be hermetically sealed and form a container having a fluid tight internal chamber, the internal chamber housing at least one drug dosage unit; a medically acceptable adhesive layer on a bottom side of the substrate member, the medical adhesive layer having a medically acceptable adhesive capable of adhering the container to skin of a person; and a releasable liner on the adhesive layer.

In one embodiment, the container member includes at least one wall having at least one flange. The at least one flange can be attached to the top side of the substrate member. The at last one wall extends outward from the top surface of the substrate member so as to provide at least one container base.

In one embodiment, the substrate member has a frangible region defined by the container member. The frangible region can be more frangible compared to the container member so that force ruptures the frangible region of the substrate member before rupturing the container member.

In one embodiment, the substrate includes a channel between the top side and bottom side. The channel can be adapted for passing the drug dosage unit therethrough upon pressure being applied to the drug dosage unit towards the channel. The adhesive layer and/or release liner can hermetically seal the channel so that the internal chamber is fluid tight.

In one embodiment, the container includes a frangible layer between the bottom side of the substrate member and the adhesive layer. The frangible layer can have a frangible region defined by the container member and covering the channel being more frangible compared to the container member so that force ruptures the frangible region before rupturing the container member.

In one embodiment, the drug dosage unit is a pill.

In one embodiment, the drug dosage unit includes an administration device containing drug.

In one embodiment, a device for making a wearable self-adhering drug dosage unit container can include: a substrate member; a container member attached to a top side of the substrate member so as to be hermetically sealed and form a container having a fluid tight internal chamber, the internal chamber housing at least one drug dosage unit; a medically acceptable adhesive layer on a bottom side of the substrate member, the medical adhesive layer having a medically acceptable adhesive capable of adhering the container to skin of a person; a releasable liner on the adhesive layer; a closable port capable of receiving the at least one drug dosage unit therethrough into the internal chamber, the closable port being in the substrate member or container member or between the substrate member and container member; and a fastener system at the closable port that is configured to close the port when fastened.

In one embodiment a method of administering a drug to a subject can include: providing a wearable self-adhering drug dosage unit container in accordance with one of the embodiments described herein; adhering the container to skin of the subject by contacting the adhesive layer to the skin; wearing the container on the skin; removing the container from the skin upon initiation of an acute medical event; removing the drug dosage unit from the container; and administering the drug to the subject.

In one embodiment, the container can include a releasable liner on the adhesive layer; and the method can then include removing the releasable liner to expose the adhesive before adhering the container to the skin.

In one embodiment, the subject has a diagnosed medical condition that is susceptible to the acute medical event, the diagnosed medical condition being treatable by the drug dosage unit.

In one embodiment, the following steps are performed by the subject: removing the container from the skin upon initiation of an acute medical event; removing the drug dosage unit from the container; and administering the drug to the subject (e.g., self-administration).

In one embodiment, the following steps are performed by a person other than the subject: removing the container from the skin upon initiation of an acute medical event; removing the drug dosage unit from the container; and administering the drug to the subject. Here, the subject can provide instructions to the person or the person may know the subject and be aware of the subject's medical complications and may be aware of the acute medical event. The subject may be aware, incapacitated, or unable to perform the method steps on their own, or the subject can assist with the performance of the method steps.

In one embodiment, the substrate member has a frangible region defined by the container member being more frangible compared to the container member, the method can then include: applying force to rupture the frangible region of the substrate member and pushing the drug dosage unit from the container.

In one embodiment, the substrate includes a channel between the top side and bottom side, the method can then include: pushing the drug dosage unit through the channel for removal from the container.

In one embodiment, the container can include a frangible layer between the bottom side of the substrate member and the adhesive layer, the frangible layer having a frangible region defined by the container member and covering the channel being more frangible compared to the container member, the method can then include: pushing on the container member to press the drug dosage unit and apply a force that ruptures the frangible region before rupturing the container member; and removing the drug dosage unit from the container through ruptured frangible region.

In one embodiment, the method can include preparing the wearable self-adhering drug dosage unit container to contain the drug dosage unit therein by placing the drug dosage unit in the container.

In one embodiment, the method can include removing the container from the skin without initiation of an acute medical event; and reapplying the container to the skin if the drug has not been administered to the subject.

In one embodiment, the acute medical event is chest pains and the drug is nitroglycerin.

In one embodiment, the subject has previously suffered a heart attack.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 is a top view showing an embodiment of the wearable self-adhering pill container invention, containing two pills, and placed on the arm or other area of skin of a person.

FIG. 2 is an enlarged top view of the embodiment of the wearable self-adhering pill container apparatus shown in FIG. 1, not yet mounted on any surface.

FIG. 3 is a bottom view of the embodiment of the wearable self-adhering pill container invention apparatus of FIG. 2, with a non-adhesive release strip being partially removed from an adhesive layer.

FIG. 4 is an enlarged cross-sectional view of the embodiment of the invention shown in FIG. 2, taken along line 4-4 thereof.

FIG. 5 is top view of another embodiment of the invention which contains a plurality of wearable self-adhering pill container apparatuses of the invention, formed together in a multiple unit configuration.

FIG. 6 shows a diagram of an embodiment of a wearable self-adhering drug dosage unit container.

FIG. 7 shows a diagram of another embodiment of a wearable self-adhering drug dosage unit container.

FIG. 8 shows a diagram of another embodiment of a wearable self-adhering drug dosage unit container.

FIG. 9 shows a diagram of an embodiment of a package having the wearable self-adhering drug dosage unit container therein.

FIG. 10 shows a diagram of an embodiment of a bottom surface of a wearable self-adhering drug dosage unit container that has an outer perimeter side without adhesive that functions as a release tab to pull from the skin.

FIG. 11 shows a diagram of a rounded embodiment of a bottom surface of a rounded or corner-less wearable self-adhering drug dosage unit container that has an outer perimeter side without adhesive that functions as a release tab to pull from the skin.

FIG. 12 shows a diagram of a kit for forming a wearable self-adhering drug dosage unit container.

FIG. 13 shows an embodiment of an adhesive applicator to apply adhesive to the bottom surface of a wearable self-adhering drug dosage unit container.

FIG. 14 shows a diagram of an embodiment of a wearable self-adhering drug dosage unit container having a port.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present invention provides an adherent drug packaging device adapted to adhere to skin of a subject. The subject can have an ailment that requires an emergency medication to treat an acute attack or disease state onset. In these types of instances, the subject may succumb to an attack at any moment without warning, and will require immediate medication, and any delay in obtaining the medication may increase the likelihood of severe complications or death. Some people with certain medical issues need to have their medications accessible at all times. For example, someone who has suffered a heart attack or is susceptible thereto (e.g., someone with a higher likelihood of having a heart attack) must have a nitroglycerin dose with them at all times so that they can administer the dose to themselves at the onset of a heart attack event. The present invention provides an adherent drug packaging device that retains the dosage in a chamber without delivering the dosage to the person. The device is adhered to the skin of the subject so that the subject can remove the dosage from the device at the onset of an attack. The device can be adhered to any part of the body, including abdomen, sides, chest, back, hips, thighs, calves, upper arms, forearms, neck, or other location. An easily accessible skin location can facilitate rapid retrieval of the dosage. For example, a blister pack may be attached to a subject's side, and they can retrieve it upon acute onset of a medical event.

The packaging can be adapted so that it is adhered to skin without causing skin irritation, by modulating the adhesive type or amount, or by providing instructions for duration of wearing the adherent drug packaging device. The packaging can be worn and replaced similar to transdermal drug delivery devices; however, the present adherent drug packaging device does not transdermally deliver the drug in view of the drug being only used during an acute onset of a medical event.

For purposes of the present invention, the term “pill” herein is understood to mean a single dose that is shape stable as an administrable unit, such as pill, tablet, capsule, or the like. A drug dosage unit can include any administrable form of a drug, such as a pill, syringe, bag, or the like that has a drug therein in a dosage unit.

While the present invention is described as a container having one or more pills therein, the pills may be substituted with any drug dosage unit. As such, reference herein to a pill may also reference a drug dosage unit, except where otherwise stated. Preferably, the drug dosage unit is a pill, however, in some instances emergency drugs need to be administered in another form for transdermal (skin patch), intravenous (syringe), inhalation (inhaler, miniature single unit inhaler), or other administration form.

A self-adhering pill container apparatus includes a substrate base member. A container member is attached to a top side of the base member. A thin adhesive layer coats a portion of a bottom side of the base member, and a non-adhesive release liner is attached to the bottom side of the thin adhesive layer. The base member and the container portion define the container's space for holding pills. Preferably, the container portion includes a container rim portion (e.g., flange) attached to the top side of the base member, and a container cup portion (e.g., at least one wall forming cup) extending upward from the container rim portion. Preferably, the base member includes a pill exit channel (e.g., drug dosage unit exit channel) that permits virtually immediate access to the medication in the container. Also, preferably, a frangible pill retention layer is included with an outer layer portion bonded to the bottom side of the base member and a frangible exit channel covering portion which covers the pill exit channel. In this respect, the container space is defined by the container cup portion, the pill exit channel, and the frangible exit channel covering portion.

The container portion can be in the form of a conventional blister pack used for pills. If desired, the thin adhesive layer can have an expiration date that can correlate to an expiration date of pills retained within the container portion.

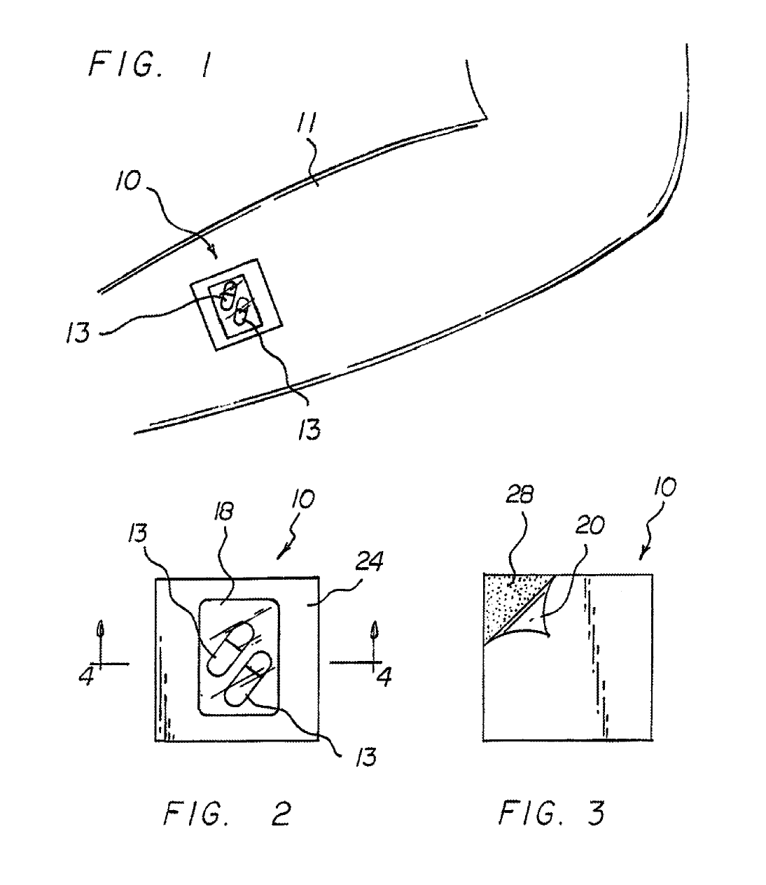

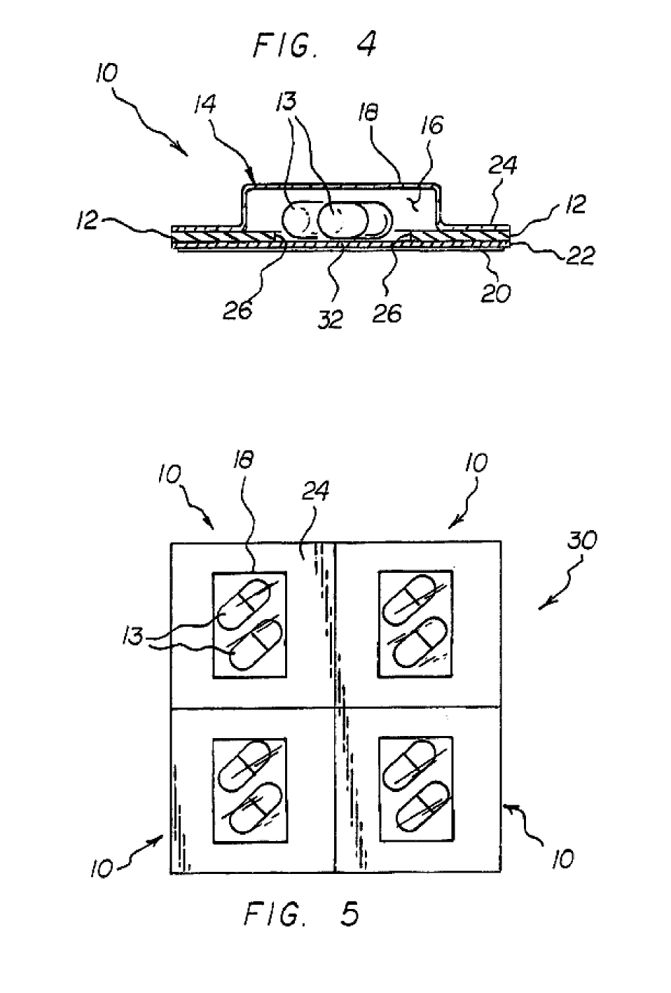

FIGS. 1-4 show a preferred embodiment of the wearable self-adhering pill container apparatus of the invention generally designated by reference numeral for the container 10. In each of the figures, reference numerals are shown that correspond to like reference numerals that designate like elements shown in other figures. FIG. 5 shows an embodiment of a package with a plurality of the containers 10 attached together.

In the preferred embodiment, the container 10 includes a substrate base member 12. A container member 14 is attached to a top side of the base member 12. As illustrated, the container member 14 includes a cover member 14a, wall member 14b, and flange member 14c that is attached to the top side of the base member 12 by any attachment, such as adhesive, melding, integration, or the like. A thin adhesive layer 28 coats at least a portion or the entirety of a bottom side of the base member 12, and a non-adhesive release liner 20 is attached to the bottom side of the thin adhesive layer 28. The base member 12 and the container member 14 define a container space 16 for containing pills 13. Preferably, the container portion 14 includes a container rim portion 24 (e.g., flange member 14c) attached to the top side of the base member 12, and a container cup 18 (e.g., cover member 14a and/or one or more wall members 14b) extending upward from the container rim portion 24. With one embodiment of the invention, to apply the container 10 to a person's skin, the non-adhesive release liner 20 is removed from the thin adhesive layer 28, and the thin adhesive layer 28 is pressed on the person's skin to retain the apparatus thereon. Then, to take a pill 13, the apparatus is pulled off of the person's skin, and the base member 12 is torn to allow access to the pills 13 inside the container portion 14.

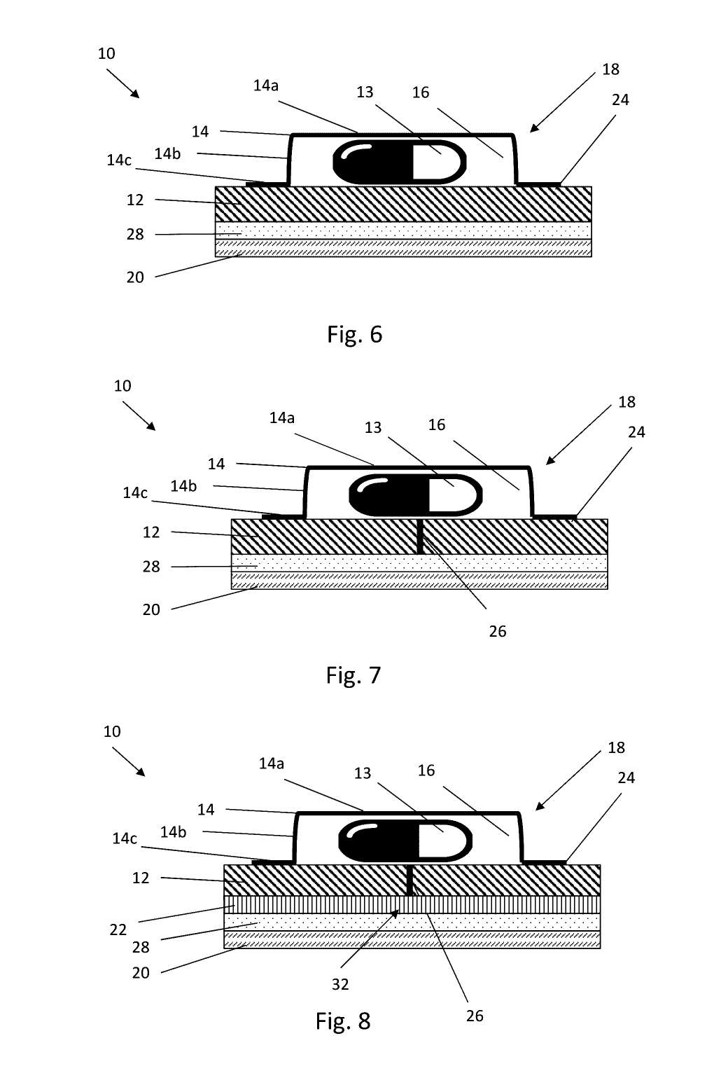

FIG. 6 shows an embodiment of the pill container 10 that has the container cup 18 attached to the base member 12 on one side and that has an adhesive layer 28 and releasable liner 20 on the other side of the base member 12. The releasable liner 20 covers all of the adhesive layer 28.

FIG. 7 shows an embodiment of the pill container 10 that has the container cup 18 attached to the base member 12 on one side and that has an adhesive layer 28 and releasable liner 20 on the other side of the base member 12. The releasable liner 20 covers all of the adhesive layer 28. The base member 12 includes a channel 26 that allows the pill to be pushed through the base member 12. While pushing, the adhesive can separate at the channel 26 to allow for the pill 13 to be pushed therethrough.

FIG. 8 shows an embodiment of the pill container 10 that has the container cup 18 attached to the base member 12 on one side and a frangible layer 22 opposite of the container cup 18. The base member 12 can include the frangible layer 22 attached thereto, such as through adhesive, melding, integration or the like. The frangible layer 22 may be a region of the base member 12 that is more frangible than the perimeter or other regions of the base member 12. The frangible layer 22 can on its other side have the adhesive layer 28 and releasable liner 20 on the other side from the base member 12. The releasable liner 20 covers all of the adhesive layer 28 and frangible layer 22. In this embodiment, the base member 12 optionally includes a channel 26 that allows the pill to be pushed through the base member 12. While pushing, the adhesive can separate at the channel 26 to allow for the pill 13 to be pushed therethrough. However, the channel 26 can be omitted when having the frangible layer 22, or both may be present.

In one aspect, the frangible layer can include a frangible exit channel covering portion 32 that covers the channel 26.

Although an embodiment of the invention need not have a base member 12 which includes a pill exit channel 26 and need not have a frangible exit channel covering portion 32, to be covered by the present invention, a preferred embodiment of the invention does include both a pill exit channel 26 and a frangible exit channel covering portion (frangible layer 22). As stated, preferably, the base member 12 includes a pill exit channel 26 that permits exit of pills 13 from the container portion 14.

Also, preferably, a frangible pill retention layer (frangible layer 22) is included and has an outer layer portion bonded to the bottom side of the base member 12 and has a frangible exit channel covering portion 32 which covers the pill exit channel 26. In this respect, the container space 16 is defined by the container cup portion 18, the pill exit channel 26, and the frangible exit channel covering portion 32.

The container member 14 and base member 12 can be in the form of a conventional blister pack used for pills 13. Such a conventional blister pack can be adapted as described herein to include the adhesive layer channel 26, frangible layer 22.

If desired, the thin adhesive layer 28 can have an expiration date that can correlate to an expiration date of pills 13 retained within the container portion 14. With this embodiment, if the pills 13 have not been used by the time of their expiration, then the apparatus can be easily removed from the person's skin because the adherence of the thin adhesive layer 28 will also have been weakened.

To fully assemble a preferred embodiment of the invention shown in FIGS. 1-8, the container member 14 is placed in an upside down orientation, and pills 13 are dropped therein. Then, the base member 12 is bonded to the container rim portion 24, such as by adhesive, melding, integration, or the like. In one aspect, the base member 12 includes a pill exit channel 26. Then, optionally, the frangible pill retention layer 22 is bonded to the bottom side of the base member 12 so that the frangible exit channel covering portion 32 of the frangible pill retention layer 22 is placed in registration with the pill exit channel 26. Then, a thin adhesive layer 28 is applied to bottom of the frangible pill retention layer 22, and a non-adhesive release liner 20 is applied to the thin adhesive layer 28 to protect the thin adhesive layer 28 when it is not in use.

For a person to carry a self-adhering pill container 10 of the invention on their body, the non-adhesive release liner 20 is peeled off of the thin adhesive layer 28, and the self-adhering pill container 10 is pressed onto a person's skin as shown in FIG. 1. The self-adhering pill container 10 is attached to the person's skin (on the person's arm 11 for illustrative purposes); by its design, the thin adhesive layer may be applied to the skin of any part of the person's body and does not require attachment in the same location that a previous pill container apparatus was adhered. As such, each subsequent container 10 can be on different locations of the skin, and even on different body parts; however, the person can continue to place the container on the same body part if that part is easy and convenient for wearing the pill container 10.

The type of adhesive used to attach the self-adhering pill container apparatus 10 to the person's skin can be the same type of adhesive that is used for medical tape or adhesive bandages. Additionally, the type of adhesive attaching medical patches, such as those for carrying and dispensing hormones, nicotine, and pain medications, to a person's skin can be used. All of the other adhesive used to adhere the layers together can be of any type, and often have more stick factor than the medically acceptable adhesive used for adherence to the skin.

When the wearer of the self-adhering pill container 10 needs to take the pills 13, the wearer pulls the self-adhering pill container 10 off one's skin. Then, as with a conventional pill containing blister pack, the person uses one hand to push on the top of the container cup portion 18, whereby the pill 13 or pills exit from the pill exit channel 26 and break through the frangible exit channel covering portion 32.

An additional embodiment of the invention is shown in FIG. 5 wherein a plurality of individual self-adhering pill containers 10 of the invention are formed together in a multiple unit configuration 30. If desired, individual self-adhering pill containers 10 can be attached by perforations to other containers. In this way, an individual self-adhering pill container apparatus 10 can be separated from the multiple unit configuration 30 by simply tearing it away along adjacent perforations.

In one embodiment, the base member alone can have the perforations, where the release liner may or may not have the perforations, and the adhesive may or may not have the perforations. When the release liners are not separated by perforations, each container may have its own release liner. When included, the frangible layer may include the perforations.

The components of the wearable self-adhering pill container apparatus can be made from inexpensive and durable plastic or paper materials.

In one embodiment, the adhesive adhering the base member to the container member may be a stronger adhesive or have more stick than the medically-acceptable adhesive on the other side of the substrate that adheres the container to the subject's skin. This allows the container to be grabbed and pulled from the skin easily so that the entire container is removed from the skin.

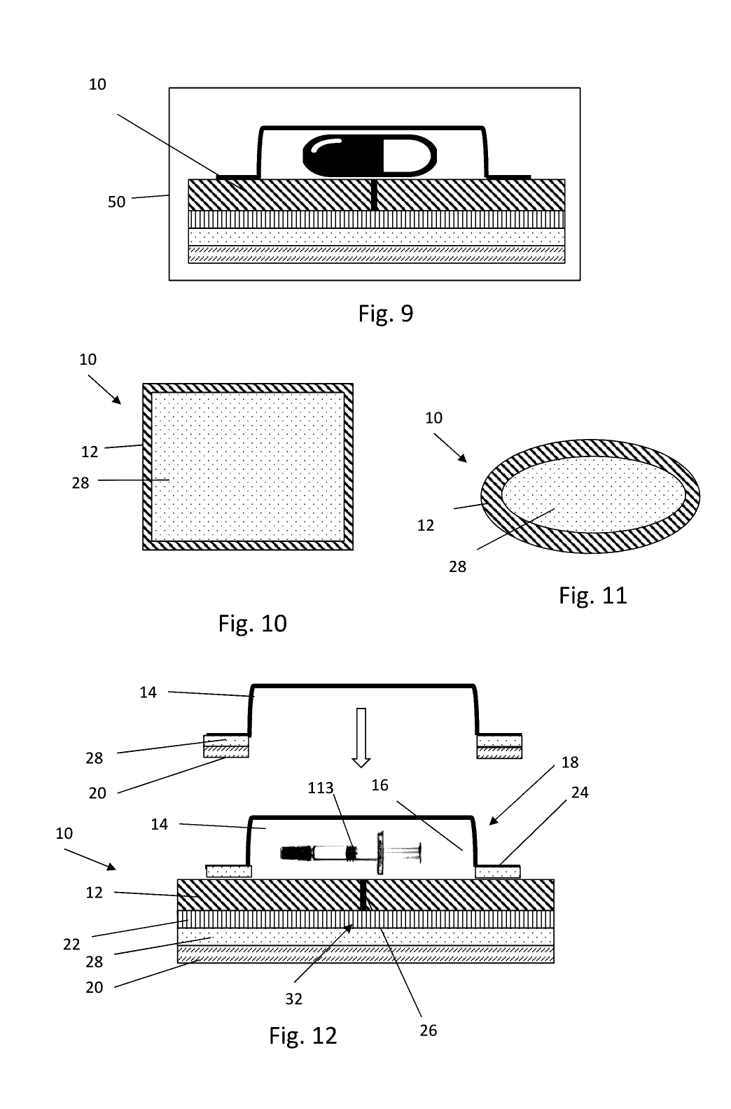

In one embodiment, the container 10 is included in a single dose package 50 as shown in FIG. 9. The container can be removed from the single dose package 50, and adhered to the skin as described herein. The package 50 can be paper, cardboard, plastic, foil, plastic, or combination thereof, rigid or flexible.

In one embodiment shown in FIG. 10, the base member 12 can have larger cross dimensions than the adhesive layer 28, so that an adhesive-free perimeter on the base member 12 (e.g., on one, two, three, or all four sides) is retained and functions as a release tab. The person can more easily pull on the adhesive free perimeter to remove the container 10 from the skin.

In one embodiment, the container can include the following: a plastic release liner coated with silicone (e.g., 5.6 mils); medical grade acrylic adhesive that has more stick (e.g., 1.4 mils); a polyester film substrate (e.g., 0.5 mil); and a polyethylene container member. The medical grade acrylic adhesive (e.g., skin adhesive) can have a strength of 27.4 N/25 mm, or the like.

In one aspect, the medically acceptable adhesive is a pressure sensitive adhesive. In one aspect, the medically acceptable adhesive is selected from acrylics, butyl rubber, ethylene-vinyl acetate, natural rubber, nitriles, silicone rubbers, styrene block copolymers, styrene-butadiene-styrene, styrene-ethylene/butylene-styrene, styrene-ethylene/propylene, or styrene-isoprene-styrene. In one aspect, the medically acceptable adhesive has a characteristic different from a characteristic of the adhesives used to adhere other layers together, and the characteristic is selected from: adhesive type, adhesive amount, adhesive stick potential, and adhesive release potential so that the container can be removed from skin before being pulled apart.

In one embodiment, the device can be devoid of corners. As such, each edge can curve into each other edge without there being a sharp point. Avoiding sharp corners can provide a container that does not poke or irritate the skin or person that is wearing the container. Such an example of rounded features is shown in FIG. 11, which illustrates a corner-less shape.

In one embodiment, the container can be prepared by the user as shown in FIG. 12. As such, a kit can include the base member 12 having the adhesive layer 28 (and optionally, the frangible layer 22) and releasable liner 20 on the rim 24 separate from the container member 14. The container member 14 can include an adhesive layer 128 and releasable liner 120. To use, the releasable liner 120 is removed to expose the adhesive layer 128, and the container member 14 is adhered to the base member 12 with the drug 113 therebetween so as to be inside the formed container. The kit may or may not include the pills. In one instance, the kit includes the pills. Here, the drug 113 is shown as being contained in a drug applicator, such as a syringe, but it could be a traditional pill.

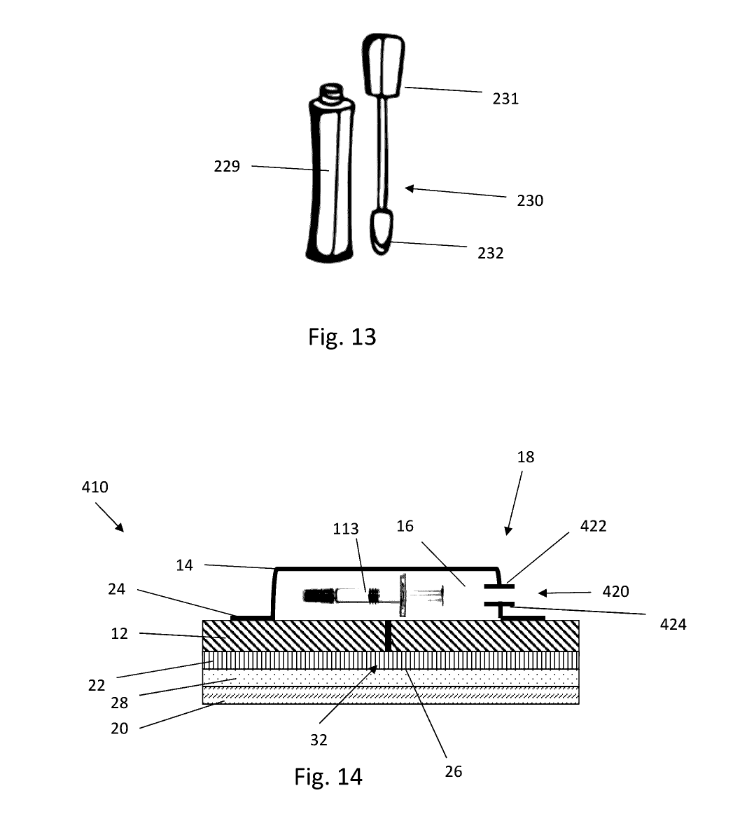

In another embodiment, the kit as shown in FIG. 13 can include an adhesive container 229 containing an adhesive with an adhesive applier lid 230 having a lid portion 231 and an applier portion 232. When the adhesive layer 28 loses enough stick that it does not still well or the container falls off the skin, the user can apply more adhesive and re-adhere the container to the skin. Also, the adhesive can be applied to the back of any pill container with a base, and then the pill container can be stuck to the skin. The adhesive container 229 can be included in any kit or used in methods described herein.

In one embodiment, the container 410 as shown in FIG. 14 includes a port 420. The port 420 allows for the drug dosage unit 311 (e.g., pill) to be inserted into the container space 16. That is, the container 410 is provided empty with instructions for the user to put their own drug dosage unit 311 inside for carrying with them. The drug dosage unit 311 can be inserted through the port 420 into the container 410. The container member 14 may also include a fastener system 422, 424 that can be sealed once the drug dosage unit 311 is therein. The fastener system 422, 424 can close by adhesive, Ziploc, tongue and groove, snap shut, or the like. The closing can be fluid tight or not. The container 410 can be applied to skin and used as described herein. Alternatively, the port 420 can be between the base member 12 and chamber member 14, with the fastener system 422, 424 appropriately located with fastener 422 on the container member 14 and the fastener 424 on the base member 12. In one instance, the fastener 422, 424 is an adhesive and release liner, where the release liner can be released so that the adhesive can adhere the container member 14 to the base member 12.

A kit can include a plurality of the containers, which may be prepackaged with the drug dosage unit or empty so that the drug dosage unit can be inserted into the container by the user.

While the channel 26 has been shown as a narrow channel, the dimensions and shape can vary from a slit to an open conduit.

The drugs can vary and examples are those that are taken during an emergency or acute medical event. Examples can include activated charcoal, adenosine, albuterol, amiodarone, acetylsalicylic acid, lorazepam, atropine sulfate, ipratropium bromide, calcium gluconate, capoten, corticosteroid, insulin, dextrose, diphenhydramine, dopamine, duoneb, epinephrine, fentanyl, furosemide, glucagon, clonazepam, trandate, lidocaine, magnesium sulfate, midazolam, morphine sulfate, naloxone, nitroglycerin, nubain, oxytocin, promethazine, zemuron, succinylcholine, vasopressin, vecuronium, ondansetron, Xanax or combinations thereof. The drugs can be provided as a pill, a liquid in a container, or in a drug administrator such as a syringe. The subject may have a disease ranging from heart condition, previous heart attack, high blood pressure, anxiety, panic disorder, PTSD, or other ailment where an acute medical event can cause harm. The drug dosage unit can be an EPI pen for an epinephrine injection to treat anaphylaxis (allergic reaction), or an insulin syringe for treating diabetes (acute onset complication).

In one embodiment, the container member is clear so that the drug dosage unit can be observed therethrough, and may be of any suitable plastic.

The substrate base member can be of various materials from semi-rigid to flexible and in either embodiment is wearable and comfortable.

In one embodiment, the drug is not part of a daily dosing regimen. That is, the subject does not know when the drug will be needed.

In one embodiment, the container member is low profile and does not protrude significantly from the skin. The dimension can be the width of a pill or other dosage unit. The container may be dimensioned depending on the desired form of the drug dosage unit.

In one embodiment, the container or kit having the container can include instructions of the method to use the container to wear on the skin and to remove before administering the drug.

In one embodiment, one of the substrate, base member, or other layer is impermeable to the drug in the drug dosage unit. In one aspect, a drug impermeable layer is located between the drug dosage unit and the adhesive.

In one embodiment, the container is not a transdermal patch and does not deliver the drug transdermally to the subject.

In one embodiment, the drug dosage unit is a transdermal patch, which is removed from the container before applying to the skin. For example, the transdermal patch can be a nicotine patch that is removed from the container and then applied when the subject feels like using nicotine. As such, the container can have the nicotine patch or any other patch that can be retrieved when needed or wanted by the client. For example, the container can be purchased with the transdermal patch as a product that can be worn by the subject, and the subject can remove the transdermal patch therefrom and apply it to their skin, where the drug in the transdermal patch cannot contact the subject's skin to deliver the drug until the patch is removed from the container and applied to the skin. For example, the container can be purchased separately from the transdermal patch and the patch can be placed into the container before the container is worn by the subject, and the subject can remove the transdermal patch therefrom and apply it to their skin, where the drug in the transdermal patch while within the container cannot contact the subject's skin to deliver the drug until the patch is removed from the container and applied to the skin.

In one embodiment, the container has the sealable port that can be resealed. As such, the subject can remove one drug dosage unit from the container while keeping one or more other drug dosage units in the container, and then the container can be reclosed.

In one embodiment, the container can be opened while still on the skin. That is, the container can be adhered to the skin, and the subject can open the container, such as through the port, and one dosage unit can be extracted therefrom, and the port resealed.

In one embodiment, the container can be work for 1 day, 2 days, 3 days, 4 days, 5 days, 6 days, 7 days, or longer without removal.

In one embodiment, the container can be removed whenever desired or needed and reapplied whenever desired or needed. Here, additional adhesive can be provided and applied to the container. Also, a kit can include double sided tape to adhere the container to the skin.

In one embodiment, the container can be removed nightly and reapplied in the morning. Here, additional adhesive can be provided and applied to the container. Also, a kit can include double sided tape to adhere the container to the skin.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is also to be understood that the terminology used herein is for the purpose of describing particular embodiments only, and is not intended to be limiting.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.

1. A wearable self-adhering drug dosage unit container comprising:
a substrate member;
a container member attached to a top side of the substrate member so as to be sealed and form a container having an internal chamber, the internal chamber housing at least one drug dosage unit;
a medically acceptable adhesive layer on a bottom side of the substrate member, the medical adhesive layer having a medically acceptable adhesive capable of adhering the container to skin of a person; and
a releasable liner on the adhesive layer.

2. The container of claim 1, the container member comprising at least one wall having at least one flange, the at least one flange attached to the top side of the substrate member, the at last one wall extending outward from the top surface of the substrate member so as to provide at least one container base.

3. The container of claim 1, the substrate member having a frangible region defined by the container member being more frangible compared to the container member so that force ruptures the frangible region of the substrate member before rupturing the container member.

4. The container of claim 1, the substrate comprising a channel between the top side and bottom side, the channel being adapted for passing the drug dosage unit therethrough upon pressure being applied to the drug dosage unit towards the channel, wherein the adhesive layer and/or release liner hermetically seals the channel so that the internal chamber is fluid tight.

5. The container of claim 4, comprising a frangible layer between the bottom side of the substrate member and the adhesive layer, the frangible layer having a frangible region defined by the container member and covering the channel being more frangible compared to the container member so that force ruptures the frangible region before rupturing the container member.

6. The container of claim 1, wherein the drug dosage unit is a pill.

7. The container of claim 1, wherein the drug dosage unit includes an administration device containing drug.

8. A device for making a wearable self-adhering drug dosage unit container, the device comprising:
a substrate member;
a container member attached to a top side of the substrate member so as to be hermetically sealed and form a container having a fluid tight internal chamber, the internal chamber housing at least one drug dosage unit;
a medically acceptable adhesive layer on a bottom side of the substrate member, the medical adhesive layer having a medically acceptable adhesive capable of adhering the container to skin of a person;
a releasable liner on the adhesive layer;
a closable port capable of receiving the at least one drug dosage unit therethrough into the internal chamber, the closable port being in the substrate member or container member or between the substrate member and container member; and
a fastener system at the closable port that is configured to close the port when fastened.

9. A method of administering a drug to a subject, the method comprising:
providing a wearable self-adhering drug dosage unit container comprising:
a substrate member;
a container member attached to a top side of the substrate member so as to be hermetically sealed and form a container having a fluid tight internal chamber, the internal chamber housing at least one drug dosage unit having a drug; and
a medically acceptable adhesive layer on a bottom side of the substrate member, the medical adhesive layer having a medically acceptable adhesive capable of adhering the container to skin of a person;
adhering the container to skin of the subject by contacting the adhesive layer to the skin;
wearing the container on the skin;
removing the container from the skin upon initiation of an acute medical event;
removing the drug dosage unit from the container; and
administering the drug to the subject.

10. The method of claim 9, the container comprising a releasable liner on the adhesive layer; and the method comprising:
removing the releasable liner to expose the adhesive before adhering the container to the skin.

11. The method of claim 9, wherein the subject has a diagnosed medical condition that is susceptible to the acute medical event, the diagnosed medical condition being treatable by the drug dosage unit.

12. The method of claim 9, wherein the following steps are performed by the subject:
removing the container from the skin upon initiation of an acute medical event;
removing the drug dosage unit from the container; and
self-administering the drug.

13. The method of claim 9, wherein the following steps are performed by a person other than the subject:
removing the container from the skin upon initiation of an acute medical event;
removing the drug dosage unit from the container; and
administering the drug to the subject.

14. The method of claim 9, wherein the substrate member has a frangible region defined by the container member being more frangible compared to the container member, the method comprising:
applying force to rupture the frangible region of the substrate member and pushing the drug dosage unit from the container.

15. The method of claim 9, the substrate comprising a channel between the top side and bottom side, the method comprising:
pushing the drug dosage unit through the channel for removal from the container.

16. The method of claim 15, the container comprising a frangible layer between the bottom side of the substrate member and the adhesive layer, the frangible layer having a frangible region defined by the container member and covering the channel being more frangible compared to the container member, the method comprising:
pushing on the container member to press the drug dosage unit and apply a force that ruptures the frangible region before rupturing the container member; and
removing the drug dosage unit from the container through ruptured frangible region.

17. The method of claim 9, comprising preparing the wearable self-adhering drug dosage unit container to contain the drug dosage unit therein.

18. The method of claim 9, comprising:
removing the container from the skin without initiation of an acute medical event; and
reapplying the container to the skin if the drug has not been administered to the subject.

19. The method of claim 9, wherein the acute medical event is chest pains and the drug is nitroglycerin.

20. The method of claim 19, wherein the subject has previously suffered a heart attack.
